(19) United States
(12) Patent Application Publication
(10) Pub. No.: US 2022/0341911 A1
(43) Pub. Date: Oct. 27, 2022

(54) DODECANE AS EXHALED BIOMARKER FOR EXERCISE-INDUCED ASTHMA IN CHILDREN

(71) Applicant: OWLSTONE MEDICAL LIMITED, Cambridge (GB)

(72) Inventors: Max Allsworth, Cambridge (GB); Marc Van Der Schee, Cambridge (GB); Tilly Woodland, Cambridge (GB); Agnieszka Smolinska, Cambridge (GB)

(21) Appl. No.: 17/765,291

(22) PCT Filed: Oct. 1, 2020
(86) PCT No.: PCT/GB2020/052399

§ 371 Date: Mar. 30, 2022

(30) Foreign Application Priority Data
Oct. 1, 2019 (GB) 1914183.7

(51) Int. Cl. G01N 33/497; G01N 33/68; A61B 5/08; A61B 5/00

(52) U.S. Cl. CPC G01N 33/497; G01N 33/6893; A61B 5/082; A61B 5/7275

(57) ABSTRACT

The invention relates to methods for predicting the response of a subject suffering from asthma or a respiratory disorder to a therapy comprising a Th2 pathway modulator and a device for use in such methods.

### INTRODUCTION

Respiratory diseases are some of the most common disorders in the world. Such respiratory diseases include conditions such as Chronic obstructive pulmonary disease (COPD), asthma, cystic fibrosis and pulmonary fibrosis. COPD, for example, affects millions of people and is responsible for extensive morbidity and mortality in the United States. COPD is a term used to describe chronic lung diseases characterized by progressive development of airflow limitation that is usually not fully reversible with medication. The common symptoms of COPD include breathlessness, wheezing and a chronic cough.

Asthma is another example of a chronic lung disease with symptoms similar to COPD, such as breathlessness and wheezing, but etiologically distinct from COPD. Asthma is a prevalent health care problem; it affects millions of people around the world. In susceptible individuals, asthma causes recurrent episodes of coughing, wheezing, chest tightness, and difficult breathing. Inflammation makes airways sensitive to stimuli such as allergens, chemical irritants, tobacco smoke, cold air and exercise. When exposed to such stimuli, airways may become swollen, constricted, filled with mucus, and hyper responsive to stimuli.

Unfortunately, many of the preventive medications have undesirable side effects, such as serious as growth limitation in children, osteoporosis, weight gain, and cataracts. As a result, the failure to properly identify the amount of inflammation in the airways, and therefore the appropriate treatment for a subjects asthmatic condition, may significantly adversely impact the subject's health. To date, however, there is no generally accepted manner of readily determining whether a given patient requires treatment, let alone what specific type of treatment should be used.

Conventionally, asthma is diagnosed by examining a number of indicators and qualitatively assessing the observed results. For example, a clinical diagnosis of asthma is often prompted by a combination of symptoms such as episodic breathlessness, wheezing, chest tightness, and coughing. However, these symptoms often occur only nocturnally and therefore are difficult for a doctor to monitor or measure. In addition, recently manifested symptoms alone are neither diagnostic indicators for asthma nor true measures of severity, so doctors must often evaluate a patient's health over long time periods before a diagnosis of asthma may be made with reasonable confidence. Because of the difficulty inherent in diagnosing asthma, doctors must use a patient's response to asthma treatments as a diagnostic tool. For example, the fact that bronchodilator treatment results in the relief of symptoms generally associated with asthma could indicate the presence of asthma. Disadvantageously, such diagnosis methods may result in the unnecessary application of asthma medications that have undesirable side effects or do not provide appropriate treatment for the underlying pathophysiological disease drivers.

There currently is no cure for asthma, but two types of treatment approaches are commonly deployed. One of these types of treatments employs quick-relief medications, such as inhaled bronchodilator therapy, which works quickly to suppress symptoms by relaxing airway smooth muscle. The other of these types of treatments employs long-term preventive medications, such as inhaled or oral steroids, leukotriene antagonists or biologicals which can prevent the onset of symptoms and attacks by controlling the underlying inflammation, thereby keeping persistent asthma under control. Many of these drugs, especially the novel biologics, target specific pathways underpinning different so-called endotypes of asthma which represent a specific type of airway inflammation. Currently there are no easy to apply tests to determine what endotype a patient belongs to, hindering the ability to prescribe the right therapy.

The current gold standard is analysis of induced sputum samples to determine inflammatory endotypes based on granulocytic cell composition, namely eosinophilic, neutrophilic, mixed granulocytic, or paucigranulocytic.

Eosinophils are granulocytic leukocytes first discovered by Heinrich Caro in 1874 and described by Paul Ehrlich in 1879 They constitute 1-4% of circulating white cells and are distinguished phenotypically by their bilobed nuclei and large acidophilic cytoplasmic granules. The pathological role of eosinophils primarily occurs in tissues and therefore a major focus has been to outline the molecular mechanisms involved in selective eosinophil recruitment to target tissues.

Eosinophilic inflammation orchestrated by allergic sensitization and T helper 2 lymphocytes (Th2)-mediated immune response is the hallmark of airway inflammation in asthma. Eosinophilic airway inflammation is considered a hallmark of type 2 inflammation in asthma.

The type 1 and type 2 immune response paradigm describes distinct immune responses that are mainly regulated by subpopulations of CD4+ T cells known as T helper 1 (TH1) and TH2 cells, respectively. TH1 cells secrete interleukin-2 (IL-2), interferon-γ (IFNγ) and lymphotoxin-α, and stimulate type 1 immunity, which is characterized by prominent phagocytic activity. By contrast, TH2 cells mainly secrete the prototypical cytokines IL-4, IL-5 and IL-13, and stimulate type 2 immunity, which is characterized by high antibody titres and eosinophilia. Type 2 immune responses are induced by parasitic helminths and are associated with atopic diseases, such as allergy and asthma. Airway type 2 immune responses are mainly mediated by eosinophils, mast cells, basophils, TH2 cells, group 2 innate lymphoid cells (ILC2s) and IgE-producing B cells. Type 2 immune responses are characteristic of allergic rhinitis in the upper airways and asthma in the lower airways.

Eosinophilic airway inflammation can be measured in the airway semi-invasively by sputum analysis and invasively by bronchoscopic sampling. Studies have shown a moderate correlation between blood eosinophil count and sputum eosinophil count (r=0.6; p<0.0001) (r=0.59; p<0.001). Using the (ROC) curves the diagnostic accuracy of a blood eosinophil count was 0.85 [95% confidence interval (CI) 0.78-0.93] [to detect a sputum eosinophilia ≥3% (George and Brightling, Therapeutic Advances in Chronic Disease, 2016, Vol. 7(1) 34-51)).

Type 2 inflammation is suppressed by glucocorticoids, which have long been the mainstay controller medication for asthma. It has been known for some time that eosinophilia predicts a favourable response to glucocorticoid treatment. Consistently in both asthma and COPD, a sputum eosinophilia is associated with a good response to corticosteroid therapy (Fahy, Nat Rev Immunol. 2015 January; 15(1): 57-65; (George and Brightling, Therapeutic Advances in Chronic Disease, 2016, Vol. 7(1) 34-51)).

Periostin, an extracellular matrix protein secreted by airway epithelial cells in response to IL-13 that regulates epithelial-mesenchymal interactions, has been associated with T2-high eosinophilic asthma. Periostin expression is increased in the asthmatic airway and may be measured in the serum. Periostin has been suggested as biomarker for Eosinophilic airway inflammation. Several studies also showed that the elevated serum periostin level predicts the response to omalizumab therapy (Tiotiu, Asthma Res Pract. 2018; 4: 10). WO2017/050527 discloses in vitro methods for diagnosing different types of airway inflammation by measuring VOCs in breath.

However, there is a need to predict and monitor response to treatment in a patient suffering from airway inflammation. There is also a need for further reliable and robust non-invasive diagnostic methods to diagnose and monitor eosinophilic airway inflammation. The invention is aimed at addressing this need.

### SUMMARY OF THE INVENTION

In a first aspect, the invention relates to an in vitro method of predicting the response of a subject suffering from a respiratory disorder to a therapy comprising a Th2 pathway modulator comprising the steps of determining the amount of dodecane and/or octanal in a sample of exhaled breath of said subject; comparing the amount of dodecane and/or octanal to a reference value and predicting that the patient will respond to the therapy when the amount measured in the sample is different compared to the reference level.

In another aspect, the invention relates to an in vitro method of monitoring the efficacy of a treatment of a subject suffering from a respiratory disorder with a therapy comprising a Th2 pathway modulator comprising the steps of

determining the amount of dodecane and/or octanal in a sample of exhaled breath of said subject;

comparing the amount of dodecane and/or octanal to a reference value and

predicting that the patient will respond to the therapy when the amount measured in the sample is different compared to the reference level.

In another aspect, the invention relates to an in vitro method of diagnosing, prognosing and/or monitoring a respiratory disorder in a subject comprising the steps of:

determining the amount of dodecane and/or octanal in a sample of exhaled breath of said subject;

comparing the amount of dodecane and/or octanal to a reference value and

predicting that the patient is likely to suffer from a respiratory disorder when the amount measured in the sample is different compared to the reference level.

In another aspect, the invention relates to an in vitro method of distinguishing eosinophilic airway inflammation in a subject from other types of airway inflammation comprising the steps of:

determining the amount of dodecane and/or octanal in a sample of exhaled breath of said subject;

comparing the amount of dodecane and/or octanal to a reference value and

predicting that the patient is likely to suffer from eosinophilic airway inflammation when the amount measured in the sample is elevated compared to the reference level.

In another aspect, the invention relates to a method of treating a respiratory disorder in a patient, comprising administering to the patient a therapeutically effective amount of a Th2 pathway inhibitor, wherein an exhaled breath sample obtained from the patient has been determined to have elevated levels of dodecane and/or octanal, compared to reference levels of dodecane and/or octanal.

In another aspect, the invention relates to a device for use in the methods.

In another aspect, the invention relates to dodecane and/or octanal for use as a biomarker.

### FIGURE

The invention is further described in the following non-limiting FIGURE.

FIG. 1: This shows the abundance of octanal in breath samples and the effect of ICS modification. Importantly, these graphs indicate a biomarker elevated in untreated individuals with asthma whilst the same biomarker is absent in those without asthma and clearly suppressed to normal levels in individuals treated with the eosinophilic inflammation suppressing drug ICS.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the invention relates to an in vitro method of predicting the response of a subject suffering from a respiratory disorder to a therapy comprising a Th2 pathway modulator comprising the steps of determining the amount of dodecane and/or octanal in a sample of exhaled breath of said subject; comparing the amount of dodecane and/or octanal to a reference value and predicting that the patient will respond to the therapy when the amount measured in the sample is different compared to the reference level.

In another aspect, the invention relates to an in vitro method of predicting the response of a subject suffering from a respiratory disorder to a therapy comprising a Th2 pathway modulator comprising the steps of

determining the amount of dodecane and octanal in a sample of exhaled breath of said subject;

comparing the amount of dodecane and octanal to a reference value and

predicting that the patient will respond to the therapy when the amount measured in the sample is different compared to the reference level.

In another aspect, the invention relates to an in vitro method of predicting the response of a subject suffering from a respiratory disorder to a therapy comprising a Th2 pathway modulator comprising the steps of

determining the amount of octanal in a sample of exhaled breath of said subject;

comparing the amount of octanal to a reference value and

predicting that the patient will respond to the therapy when the amount measured in the sample is different compared to the reference level

In another aspect, the invention relates to an in vitro method of monitoring the efficacy of a treatment of a subject suffering from a respiratory disorder with a therapy comprising a Th2 pathway modulator comprising the steps of

determining the amount of dodecane and/or octanal in a sample of exhaled breath of said subject;

comparing the amount of dodecane and/or octanal to a reference value and

predicting that the patient will respond to the therapy when the amount measured in the sample is different compared to the reference level.

In another aspect, the invention relates to an in vitro method of monitoring the efficacy of a treatment of a subject suffering from a respiratory disorder with a therapy comprising a Th2 pathway modulator comprising the steps of

determining the amount of dodecane and octanal in a sample of exhaled breath of said subject;

comparing the amount of dodecane and octanal to a reference value and

predicting that the patient will respond to the therapy when the amount measured in the sample is different compared to the reference level.

In another aspect, the invention relates to an in vitro method of monitoring the efficacy of a treatment of a subject suffering from a respiratory disorder with a therapy comprising a Th2 pathway modulator comprising the steps of

determining the amount of octanal in a sample of exhaled breath of said subject;

comparing the amount of octanal to a reference value and

predicting that the patient will respond to the therapy when the amount measured in the sample is different compared to the reference level.

In another aspect, the invention relates to an in vitro method of diagnosing, prognosing and/or monitoring a respiratory disorder in a subject comprising the steps of:

determining the amount of dodecane and/or octanal in a sample of exhaled breath of said subject;

comparing the amount of dodecane and/or octanal to a reference value and

predicting that the patient is likely to suffer from a respiratory disorder when the amount measured in the sample is different compared to the reference level.

In another aspect, the invention relates to an in vitro method of diagnosing, prognosing and/or monitoring a respiratory disorder in a subject comprising the steps of:

determining the amount of dodecane and octanal in a sample of exhaled breath of said subject;

comparing the amount of dodecane and octanal to a reference value and

predicting that the patient is likely to suffer from a respiratory disorder when the amount measured in the sample is different compared to the reference level.

In another aspect, the invention relates to an in vitro method of diagnosing, prognosing and/or monitoring a asthma and/or eosinophilic airway inflammation in a subject comprising the steps of:

determining the amount of dodecane and octanal in a sample of exhaled breath of said subject;

comparing the amount of dodecane and octanal to a reference value and

predicting that the patient is likely to suffer from a respiratory disorder when the amount measured in the sample is different compared to the reference level.

In another aspect, the invention relates to an in vitro method of distinguishing eosinophilic airway inflammation in a subject from other types of airway inflammation comprising the steps of:

determining the amount of dodecane and/or octanal in a sample of exhaled breath of said subject;

comparing the amount of dodecane and/or octanal to a reference value and

predicting that the patient is likely to suffer from eosinophilic airway inflammation when the amount measured in the sample is elevated compared to the reference level.

In another aspect, the invention relates to a method of treating a respiratory disorder in a patient, comprising administering to the patient a therapeutically effective amount of a Th2 pathway inhibitor, wherein an exhaled breath sample obtained from the patient has been determined to have elevated levels of dodecane and/or octanal, compared to reference levels of dodecane and/or octanal.

In one embodiment of the aspects above, the respiratory disease is asthma and/or eosinophilic airway inflammation.

Exhaled breath contains low concentrations of various volatile organic compounds (VOCs) produced by the body. These are believed to reflect endogenous metabolic processes at the tissue level, such as inflammation and oxidative stress. VOCs present in exhaled breath have been shown to be able to discriminate between various lung pathologies (WO2017/050527). Thus, VOC biomarkers in breath can offer a non-invasive, robust and reproducible way of diagnosing and monitoring respiratory disease. The inventors have found that dodecane and octanal, either alone or in combination, can be used as biomarkers in exhaled breath to indicate, in a subject suffering from a respiratory disease such as asthma, the response to treatment, in particular in response to treatment with a Th2 pathway modulator, such as a steroid. Also, these markers enable the diagnosis of a respiratory disease in particular eosinophilic airway inflammation.

The term “volatile organic compounds” (abbreviated VOC, VOCS or VOCs) refers to organic chemicals, or derivatives thereof, present in exhaled breath from a subject. The VOCs of the various aspects of the invention are selected from dodecane and/or octanal. According to the various methods described herein, either dodecane or octanal can be used or dodecane and octanal can be used in combination.

Dodecane is also known as n-Dodecane, 112-40-3, Dihexyl, Bihexyl, adakane 12 or duodecane. It is a liquid alkane hydrocarbon with the chemical formula C12H26 and has a molecular weight of 170.33 g/mol. Octanal has a molecular formula of C8H16O and a molecular weight of 128.21 g/mol.

“Eosinophilic airway inflammation” describes one subtype of asthma. Other subtypes are neutrophilic, mixed granulocytic and paucigranulocytic airway inflammation. Usually sputum eosinophil cell count cut-offs are used for diagnosis. which depend on guidelines. Eosinophilic airway inflammation can for example be characterised by a correlation between blood eosinophil count and sputum eosinophil count (r=0.6; p<0.0001) (r=0.59; p<0.001).

A “subject” as used herein refers to a test subject, e.g. a mammalian subject, preferably a human. In one embodiment, a sample of exhaled breath is obtained from the subject for the purpose of diagnosing or screening the presence/absence of an airway inflammation, e.g. eosinophilic airway inflammation or making a prognosis as to the likelihood that the subject will develop an airway inflammation, e.g. eosinophilic airway inflammation. In another aspect, a sample of exhaled breath is obtained from the subject for the purpose of assessing or determining a treatment. In that case, the subject is preferably one that has been diagnosed with an airway inflammation. The subject may be male or female. The subject may be an infant, a toddler, a child, a young adult, an adult or a geriatric. The subject may exhibit one or more symptoms of aspiratory disorder, such as asthma. In some aspects, the subject may exhibit one or more symptoms of eosinophilic airway inflammation. Thus, in some aspects, the method can differentiate between subjects with and without asthma.

As used herein, a “healthy subject” is defined as a subject that does not have a diagnosable asthma, a respiratory disease and/or eosinophilic airway inflammation.

A “test subject value” is the value obtained in a test subject, i.e. a subject that is being assessed. The test value is the concentration of the VOC, i.e. dodecane and/or octanal, that is measured in exhaled breath.

As used herein, “reference value”, “baseline” or “threshold value” means a value determined by performing the testing method on one or more, preferably a plurality of reference subjects. A reference subject can be a healthy subject or a subject diagnosed with asthma, a respiratory disease or eosinophilic airway inflammation. Preferably, the test subject is a subject that suffers from a respiratory disease such as asthma and has not received treatment. The test subject shares characteristics with the reference subject, e.g. if the test subject is a child or adolescent, then the reference subject is a child or adolescent. In the methods of the invention, the reference subject can be selected from one of the following:
An untreated patient with a respiratory disease;
The patient him or herself (own reference);
Asthma patient with different inflammatory subtype or
A patient without a respiratory disease.

A “likelihood of eosinophilic airway inflammation” means that the probability that the eosinophilic airway inflammation disease state exists in the subject specimen is about 50% or more, for example 60%, 70%, 80% or 90%.

The term “monitoring” as used herein generally refers to the monitoring of asthma, a respiratory disease and/or eosinophilic airway inflammation progression or regression over time (e.g. between two or more sample of exhaled breath from a subject, taken at different time intervals), preferably following treatment. Also encompassed by this term is the evaluation of treatment efficacy using the methods of the present invention.

The terms “diagnosing” or “diagnosis” generally refer to the process or act of recognizing, deciding on or concluding on a disease or condition in a subject on the basis of symptoms and signs and/or from results of various diagnostic procedures (such as, for example, from knowing the presence, absence and/or amount of dodecane and/or octanal characteristic of the diagnosed disease or condition).

“Prognosis” refers to an assessment of the likelihood that the subject will experience a worsening of an existing disease or that the subject will develop a respiratory disease, asthma and/or eosinophilic airway inflammation. A good prognosis of the diseases or conditions may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions may generally encompass anticipation of a substandard recovery and/or unsatisfactorily slow recovery, or to substantially no recovery or even further worsening of such.

“Therapeutic treatment” refers to treatment of a respiratory disease, asthma and/or eosinophilic airway inflammation. This includes Th2 inhibitors, such as asthma treatments known in the art, such as inhaled corticosteriod (ICS) therapy as well as other therapies, such as antibody therapy. In one embodiment of the methods, the treatment is with ICS.

The terms “concentration”, “amount”, “quantity”, or “level” are used herein interchangeably and are generally well understood in the art. The terms as used herein may particularly refer to an absolute quantification of dodecane and/or octanal in a sample, or to a relative quantification of dodecane and/or octanal in a sample, i.e., relative to another value such as relative to a reference value as taught herein, or to a range of values indicating a base-line expression of dodecane and/or octanal. These values or ranges can be obtained from a single subject or from a group of subjects.

The term “respiratory disorder” refers to disorder of the airways, more particularly airway inflammation, such as asthma, more particularly eosinophilic airway inflammation.

In one embodiment, the methods of the invention comprise collecting a breath sample from a test subject and optionally from a reference subject. The breath sample can include air exhaled from one or more different parts of the subject's body (e.g. nostrils, pharynx, trachea, bronchioles, alveoli etc.). A sample of exhaled breath may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably collection of a sample of exhaled breath is performed in a minimally invasive or a non-invasive manner. For the collection of a breath sample and methods of measurement, the device and methods described in WO2017/187120 or WO2017/187141 (both publications are hereby incorporated by reference) can be used.

In embodiments that include collecting inhaled air or air that will be inhaled, the sample of inhaled breath may simply be a sample of ambient air in the environment that is representative of air inhaled by the subject. In another embodiment, the subject may breathe through a breathing tube, a sample of which may be collected in the breath collector. Measurements may be taken in situ or perhaps in a gas blender or mixer that provides a mixed source of gas to a subject. In one embodiment, if inhaled air is being collected, both inhaled and exhaled breath may be sampled through the same breath collector. For example, the breath collector may first sample the inhaled breath (e.g., ambient air, air inside an incubator, etc.), analyze the inhaled breath for gas concentrations of one or more gaseous constituents, and then sample and analyze the exhaled breath of the subject. In an alternate embodiment, there may be multiple breath collectors: one for inhaled breath, and another for exhaled breath. In other embodiments, only the exhaled or expired air is sampled through the breath collector. VOCs are measured by sensors in the device and these may be connected to a processor. This may be configured for using the difference value of each of the gaseous compounds to normalize the concentration of the first gaseous compound in relation to the second gaseous compound that is indicative of the subject's breathing pattern. To this end, the processor calculates a ratio of the concentration of the first gaseous compound to the second gaseous compound to generate a normalized concentration of the first gaseous compound.

In one aspect, the invention involves establishing a reference value from a reference subject.

The methods involve determining the concentration of dodecane and/or octanal in the breath sample and then comparing the concentration to a baseline value or range. Typically, the baseline value is representative of the concentration of dodecane and/or octanal in a reference subject, e.g. in a person suffering from the disease such as asthma. Variation of levels of dodecane and/or octanal from the baseline range as explained herein indicates that the patient has a respiratory disease, asthma and/or eosinophilic airway inflammation or is at risk of (worsening of) a respiratory disease, asthma and/or eosinophilic airway inflammation.

For example, an increased concentration of dodecane and/or octanal in exhaled breath of a test subject compared to a baseline value in a reference individual, wherein the reference is from a subject that has received treatment or does not suffer from the disease indicates the presence of a respiratory disease, asthma and/or eosinophilic airway inflammation and/or indicates that the subject is likely to show responsiveness to steroid treatment.

In other words, in a subject with untreated disease, the levels of dodecane and octanal are high. In a subject with an absence of disease or in an individual with treated disease, the levels of dodecane and octanal are low.

Of particular interest according to the methods is assessing disease in a subject where the reference is from the dame subject. In other words, the method is used to assess the progression of disease in the same subject by assessing a change in the biomarker. A change from the baseline value prior to treatment can be used for a subject to assess if treatment is needed or to assess effectiveness of a treatment schedule. The method may include the additional step of determining a treatment schedule.

The algorithm used to calculate a risk assessment score in a method disclosed herein may group the concentration values of dodecane and/or octanal, and the risk score can be derived from any algorithm known in the art. The algorithms are sets of rules for describing the risk assessment of a respiratory disease, asthma and/or eosinophilic airway inflammation using expression of the panel of genes described herein. The rule set may be defined exclusively algebraically but may also include alternative or multiple decision points requiring domain-specific knowledge, expert interpretation or other clinical indicators. Many algorithms that can provide different risk assessments can be developed using concentration profiles of dodecane and/or octanal. For example, the risk scores of an individual may be generated using a Cox proportional hazard model. An individual's prognostic categorization can also be determined by using a statistical model or a machine learning algorithm, which computes the probability of recurrence based on the individual's concentration of dodecane and/or octanal.

Based on the determination of a risk, individuals can be partitioned into risk groups (e.g., tertiles or quartiles) based on a selected value of the risk score, where all individuals with values in a given range can be classified as belonging to a particular risk group. Thus, the values chosen will define risk groups of patients with respectively greater or lesser risk. Longitudinal samples can be assessed to assess the risk for a specific individual of experiencing an exacerbation of disease. This risk would then be variable over time.

The concentration of dodecane and/or octanal can be measured using methods known in the art. The concentration as used herein means the content or mass of the dodecane and/or octanal in exhaled breath as expressed, for example in grams/litre (g/l). In one embodiment, concentration is measured over time, for example by measuring the kinetics of the clearance. For example, dodecane and/or octanal concentration can be measured in the same breath sample at the same time or at different times. Also, different fractions breath can be analysed, i.e. lower and upper breath samples. In one embodiment, the sample analysed is in lower breath.

In one embodiment, the concentration or amount of the dodecane and/or octanal may be determined in absolute or relative terms in multiple breath samples, e.g. in a first breath sample (collected at a first time period) and in a second and/or further breath sample (collected at a later, second or further time period), thus permitting analysis of the kinetics or rate of change of concentration thereof over time.

In one embodiment, the methods of the invention further comprise establishing a test subject value for dodecane and/or octanal concentration.

In one embodiment, the methods of the invention further comprise comparing the test subject value to one or more reference value. In one embodiment, said reference value is from a subject suffering from asthma, for example the median value of a cohort of asthma sufferers. In another embodiment, the reference value is from subjects diagnosed with asthma.

In one embodiment, the reference value is a healthy subject value corresponding to values calculated from healthy subjects. In one embodiment, the presence of one or more subject values at quantities greater than their respective range of healthy subject values indicates a substantial likelihood of a respiratory disease, asthma and/or eosinophilic airway inflammation in the test subject.

In one embodiment, when an appropriate reference is indicative of a subject being free of a respiratory disease, asthma and/or eosinophilic airway inflammation, a detectable difference (e.g., a statistically significant difference) between the value determined from a subject in need of characterization or diagnosis of a respiratory disease, asthma and/or eosinophilic airway inflammation and the appropriate reference may be indicative of a respiratory disease, asthma and/or eosinophilic airway inflammation in the subject. In one embodiment, when an appropriate reference is indicative of a respiratory disease, asthma and/or eosinophilic airway inflammation, a lack of a detectable difference (e.g., lack of a statistically significant difference) between the value determined from a subject in need of characterization or diagnosis of EIA and the appropriate reference may be indicative of a respiratory disease, asthma and/or eosinophilic airway inflammation or absence of a respiratory disease, asthma and/or eosinophilic airway inflammation in the subject.

Thus, in one aspect, the methods include detecting the concentration of dodecane and/or octanal in exhaled breath from the subject, and diagnosing the subject as having a likelihood or increased risk of a respiratory disease, asthma and/or eosinophilic airway inflammation disease state if the level of one or more of dodecane and/or octanal is different from the reference subject value.

Thus, the methods may further comprise the steps of:

a) Comparing the amount of dodecane and/or octanal in exhaled breath with a reference value, said reference value representing a known diagnosis, prognosis and/or monitoring status of a respiratory disease, asthma and/or eosinophilic airway inflammation or representing a reference value as defined herein;

b) Finding a deviation or no deviation of the amount of dodecane and/or octanal from said value; and

c) Attributing said finding of deviation or no deviation to a particular diagnosis, prognosis and/or monitoring status of a respiratory disease, asthma and/or eosinophilic airway inflammation, in the subject.

The term “deviation of the amount” refers either to elevated or reduced amounts of dodecane and/or octanal in a sample of exhaled breath from a subject compared to a reference value. By “elevated amounts” we mean that the amount of dodecane and/or octanal in a sample of exhaled breath from a subject is statistically higher than the reference value. By “reduced amounts” we mean that the amount of dodecane and/or octanal in a sample of exhaled breath from a subject is statistically lower than the reference value. The amount may be considered to be statistically higher or lower if its value differs from a predetermined threshold value. This threshold value can, for example, be the median of the amount of dodecane and/or octanal determined in a sample of exhaled breath from a population of healthy subjects.

The term “no deviation of the amount” refers to similar or unchanged amounts of dodecane and/or octanal of the invention in a sample of exhaled breath from a subject compared to a reference value. By “similar or unchanged level” is meant that the difference of the amount of dodecane and/or octanal in a sample of exhaled breath from the subject compared to the reference value is not statistically significant. Preferably, the reference value is obtained in samples of exhaled breath obtained from one or more subjects of the same species and the same sex and age group as the subject in which a respiratory disease, asthma and/or eosinophilic airway inflammation is to be determined, prognosed or monitored. Alternatively, the reference value may be a previous value for the amount of dodecane and/or octanal obtained in a sample of exhaled breath from a specific subject. This kind of reference value may be used if the method is to be used for monitoring a respiratory disease, asthma and/or eosinophilic airway inflammation, e.g. over time, or to monitor the response of a subject to a particular treatment.

The method may also comprise determining a risk score of the subject based on the concentration of dodecane and/or octanal in the sample and using the risk score to provide a prognosis for the subject, wherein the risk score is indicative of said prognosis.

A sample of exhaled breath may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of dodecane and/or octanal in a sample of exhaled breath from a subject may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Electronic Nose device, quartz crystal microbalance or chemically sensitive sensors.

The amount of dodecane and/or octanal in a sample of exhaled breath from a subject may be determined using thermal desorption-gas chromatography-time of flight-mass spectrometry (GC-tof-MS). In certain embodiments, breath of the subject is collected in an inert bag, then the content of the bag is transported under standardised conditions onto desorption tubes and VOCs are analyzed by thermally desorbing the content of the tube and then separated by capillary gas chromatography. Then volatile organic peaks are detected with MS and identified using for example a library, such as the National Institute of Standards and Technology. Thermal desorption may be performed at the GC inlet at a temperature of, e.g., about 200-350° C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine one or more VOCs from a sample of exhaled breath from a subject.

In one embodiment, the method comprises collecting different selected exhaled breath samples, or fractions thereof, on a single breath sample capture device, the method comprising the steps of:

(a) collecting a first exhaled breath sample by contacting the sample with a capture device comprising an adsorbent material;

(b) collecting a second exhaled breath sample by contacting the second sample with said capture device, wherein the first and second exhaled breath samples are caused to be captured on the capture device in a spatially separated manner.

In some embodiments, the capture device comprises an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. In one embodiment, the capture device comprises both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

The methods of the invention may further include the step of selecting a treatment for a respiratory disease, asthma and/or eosinophilic airway inflammation following the diagnosis. The methods may then further include administering said treatment to said subject. The treatment may be a steroid treatment, such as ICS or an antibody based therapy.

The invention also relates to a method for monitoring the progression of a respiratory disease, asthma and/or eosinophilic airway inflammation, comprising assessing the activity of dodecane and/or octanal in exhaled breath of the subject.

In one embodiment, the subject has undergone treatment. This treatment refers for example to ICS treatment. In one embodiment, the person is not undergoing treatment, such as ICS treatment.

The invention also relates to the use of dodecane and/or octanal as a biomarker of a respiratory disease, asthma and/or eosinophilic airway inflammation. In particular, the invention also relates to the use of dodecane and/or octanal in a method for diagnosing a respiratory disease, asthma and/or eosinophilic airway inflammation or monitoring the progression of a respiratory disease, asthma and/or eosinophilic airway inflammation. For example, the invention also relates to the use of dodecane and/or octanal in a method for diagnosing a respiratory disease, asthma and/or eosinophilic airway inflammation or monitoring the progression of a respiratory disease, asthma and/or eosinophilic airway inflammation comprising determining the amount of dodecane and/or octanal in a sample of exhaled breath of a subject.

The invention also relates to a method of treating a subject having a respiratory disease, asthma and/or eosinophilic airway inflammation comprising determining the amount of dodecane and/or octanal in a sample of exhaled breath of a subject.

The method may also comprise determining a risk score of the subject based on the concentration of dodecane and/or octanal in the sample; using the risk score to provide a prognosis for the subject, wherein the risk score is indicative of said prognosis; and treating the subject having a high risk assessment with a therapeutic therapy.

Additionally, provided herein are methods for monitoring efficacy and appropriate dosing of a treatment for a respiratory disease, asthma and/or eosinophilic airway inflammation in a subject comprising determining the amount of VOC in a sample of exhaled breath of a subject wherein the VOC is selected from dodecane and/or octanal. In one embodiment, the treatment is with a Th2 pathway modulator, e.g. steroids. This method facilitates personalised biomarker-specific titration of corticosteroid therapy. The method can predict exacerbation risk in patients that receive ICS treatment. Adjusting corticosteroid therapy using biomarker scores as described herein will lead to more appropriate corticosteroid dosing in severe asthma, with no increase in exacerbation risk and a reduction in corticosteroid load compared to standard care.

The invention also relates to a system for detecting, diagnosing or monitoring a respiratory disease, asthma and/or eosinophilic airway inflammation comprising determining the amount of one or more VOC in a sample of exhaled breath of a subject wherein the VOC is selected from dodecane and/or octanal wherein said system comprises a device for capturing a breath sample from a patient.

In one embodiment, the system includes a device for capturing a breath sample as described in WO2017/187120 or WO2017/187141. The device in WO2017/187120 comprises a mask portion which, in use, is positioned over a subject's mouth and nose, so as to capture breath exhaled from the subject. The exhaled breath samples are fed into tubes containing a sorbent material, to which the compounds of interest adsorb. After sufficient sample has been obtained, the sorbent tubes are removed from the sampling device and the adsorbed compounds desorbed (typically by heating) and subjected to analysis to identify the presence and/or amount of any particular compounds or other substances of interest. The preferred analytic technique is field asymmetric ion mobility spectroscopy (abbreviated as “FAIMS”). The method in WO2017/187141 refinement of the method described in WO2017/187120 is disclosed in WO2017/187141. In that document, it is taught to use breath sampling apparatus substantially of the sort described in WO2017/187120, but in a way such as to selectively sample desired portions of a subject's exhaled breath, the rationale being that certain biomarkers or other analytes of interest are relatively enriched in one or more fractions of the exhaled breath, which fractions themselves are relatively enriched in air exhaled from different parts of the subject's body (e.g. nostrils, pharynx, trachea, bronchioles, alveoli etc).

The invention also relates to a device for use in the methods described herein.

The invention also relates to a kit comprising a system for detecting, diagnosing or monitoring a respiratory disease, e.g. asthma and/or eosinophilic airway inflammation wherein said system comprises a device for capturing a breath sample from a patient.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### Examples

The invention is further described in the following non-limiting examples.

A study was undertaken in children with paediatric asthma. Lower airway (alveolar enriched) and upper airway (bronchial enriched) breath samples were collected using the ReCIVA Breath Sampler from children. Breath samples were analysed via thermal desorption-gas chromatography-mass spectrometry (TDGC-MS) to determine their volatile organic compound composition.

Across all lower and upper airway samples collected from 21 patients with Exercise Induced Asthma (EIA) and 25 asthma control patients. Several molecular features (MFs)—breath compound surrogates—were found. In order to assess the potential impact of inhaled corticosteroid (ICS) on breath biomarkers, we performed a two-way ANOVA and found two compounds, dodecane and octanal, with evidence of ICS effect modification.

This study provides evidence of breath biomarkers that respond to ICS treatment and can be used to diagnose eosinophilic asthma.

### Methodology

### Samples: Sample Collection

Breath samples were collected prior to exercise challenge using a ReCIVA Breath Sampler (Owlstone Medical Ltd.). The ReCIVA can sample multiple fractions of tidal breath simultaneously; it monitors subjects' breathing pattern in real time using CO2 and pressure sensors, triggering sampling pumps to collect specific breath fractions. For this study two types of breath samples were collected, both consisting of mixed exhaled air from tidal breathing. One fraction consisted of breath dominated by air from the upper airways. This fraction maximises the opportunity for discovery of biomarkers directly from the bronchial epithelium and bronchial mucosa. The other fraction consisted of breath dominated by end tidal breath, which reflects the alveolar air VOC concentration and therefore maximises discovery of blood-based biomarkers. During the procedure, two samples of 500 mL of lower airway exhaled breath and two samples of 500 mL of upper airway exhaled breath were collected onto separate pairs of Tenax TA/Carbograph 5TD sorbent tubes (Markes International).

### Sample Size and Population

The population consisted of 46 children aged between 4 and 14 years old with respiratory symptoms and suspected of having EIA. All participants underwent an exercise challenge test to determine if they had EIA. Here, exercise induced asthma is defined as a FEV1 decrease >13% (if on ICS) or >20% (if not on ICS) after exercise challenge. Of study participants, 21 had exercise induced asthma and 25 did not. Patients without EIA will be referred to as “asthma controls” or “asthma control patients” throughout the text.

### Sample Analysis

Samples were analysed using the Breath Biopsy platform in the Breath Biopsy Laboratory (Owlstone Medical Ltd.). Samples were purged to remove excess water and desorbed using a TD100-xr thermal desorption autosampler (Markes International) and transferred onto a VF5 ms column (60 m×0.25 mm×0.25 um; CP8960 Agilent Technologies) using 1:2 split injection. Chromatographic separation was achieved via a programmed method (50-310° C. in 60.3 min. at 2.0 mL/min.) on a 1310 oven (Thermo Fisher Scientific) and mass spectral data acquired using an electron impact ionization time-of-flight (EI-TOF) BenchTOF HD mass spectrometer (Markes International). See Table 1 for an example of a routine analysis

TABLE 1
Routine analysis sequence layout
TD-GC-TOF System Checks
Gases pressure check
Air water check
Cold trap Blank
Check the Cold trap data
System blank
T-SC (Sensitivity standard)
TF-QC-DC (QC standard)
Air water check
TOF Patient samples sequences
TF-QC-DC (QC standard)
Max of 8 × sample tubes
(4 Patient samples-1 tube was upper breath
and the second tube was lower breath from
the same patient, both tubes analysed consecutively)
TF-QC-DC (QC standard)

### Data Analysis

### Feature Extraction

TD-GC-MS chromatograms were converted into features lists for statistical analysis. The process of extracting features is identifying a set of characteristics indicative of a compound and aligning them across all samples to ensure that the same feature is consistently identified and extracted when present in any sample from the data set. The peak area of the most robust ion (known as the quantifier ion) provides a measure of the abundance of the compound in the sample. Samples were batched into groups with minimal variability in retention time (RT). These batches were subsequently RT corrected to align samples for the duration of the study. Initial deconvolution was performed with Profinder (Agilent). Masshunter Quantitative Analysis software (Agilent) allowed the extracted ions from all 46 samples to be simultaneously inspected for measures of feature consistency and quality.

This processing resulted in a set of multiple features, termed molecular features (MFs), suitable for comparison across the sample set. The MFs were matched against the National Institute of Standards and Technology (NIST) standard reference database (2017), a library of over 200,000 compounds, and selected based on the highest Match factor (a measure of how well the mass spectrum matches spectra of known standards) to provide a tentative identification. A match factor >70-80% represents a hit with a good probability of reflecting the true structure or chemical class, but all tentative IDs require structural elucidation analysis or comparison to a true standard to establish the real ID. After removing features with high NIST hits as siloxane contaminants, features were imported into the Masshunter Quantitative Analysis software (Agilent Technologies) and integrated.

### Statistical Analysis

The relationship between MF variation and demographic and clinical features was explored using Principle Component Analysis (PCA), an approach to visually inspect the underlying structure of the data. When features are correlated, PCA identifies the orthogonal directions (principal components (PCs)) in the data that explain the most variance. Importantly, PCA is unsupervised as the transformation is conducted without reference to groups labels. The first PC is chosen to account for as much of the variability in the data as possible and the subsequent PCs account for as much of the variability in the data as possible under the constraint that it is orthogonal to the preceding components. As the majority of the variance is typically limited to the first few PCs, the first two principal components were plotted and coloured by the demographic feature of interest to investigate the underlying structure of the data; outliers can also be identified from such plots. To investigate whether the peak area of each MF differed between the EIA and asthma control patients, we used the Mann-Whitney U test, a two-sample non-parametric test. The analysis included all MFs and did not require the MFs to be normally distributed. To control for multiple testing, a Bonferroni threshold (α=0.05) was used. The MF unadjusted p-values along with their estimated average fold-changes (EIA/asthma control) were visualized in a volcano plot, which is a scatter plot of −log 10 unadjusted p-values (significance) versus log 2 fold-change. The distribution of individual MFs were summarised using boxplots where appropriate.

To test whether ICS treatment modifies the effect of EIA on the MFs identified, two-way analysis of variance (two-way ANOVA) was conducted to test for effect modification. A two-way ANOVA was used in the absence of an equivalent non-parametric test. An unadjusted interaction pvalue <0.2 was considered to provide some evidence of an ICS effect modification. Subgroup analysis was then performed on all MFs. Finally, a multivariable approach was taken to see whether a combination of MFs could classify EIA patients better than any single MF. Linear Discriminant Analysis (LDA) with shrinkage calculated from Ledoit-Wolf lemma1 was used to determine how well the MFs can be used to distinguish samples from EIA vs asthma control patients. LDA was selected because of the relatively small sample size and the risk of overfitting, which is much higher in non-parametric approaches such as random forest. To further reduce the risk of overfitting, the classification pipeline also included an ANOVA F-test based feature selection step. Only the M most significant MFs from the training set were used to construct the LDA model. The optimal M was defined to be the one that maximizes the mean AUC (receiver operating characteristic area under the curve) across all folds generated from repeated stratified K-fold cross-validation2. The number of folds was defined such that each left-out set contains at least two samples from the least-represented class. The optimal M was then used in a leave-one out cross validation to estimate the overall AUC.

A permutation test with five-thousand label permutations was used to assess the statistical significance of the overall AUC. In each permutation, an AUC of the LDA pipeline (with optimized M) was calculated using a leave-one-out cross validation. This generated an empirical null distribution of AUCs, which was then used to calculate the p-value.

### Results

### Lower and Upper Breath Samples for All Patients

In order to maximize the chances of finding breath compounds associated with EIA, two breath fraction samples were collected from each patient, upper airway dominated breath and lower airway dominated breath. Previous work has established that the concentration and presence of breath compounds varies over the course of an exhalation (van den Velde S, et al. “Differences between Alveolar Air and Mout Air.” Anal. Chem. 79. 2007). The general hypothesis is that early exhalation contains more exogenous compounds as well as compounds generated by cells in the upper airway while late exhalation contains more systemic, endogenous compounds as well as compounds produced by lower airway cells. However, research is still lacking on exact origins of breath compounds, and, for discovery work, it is unknown which fraction might contain putative biomarkers.

Breath samples from both fractions were successfully collected and analysed for 46 patients, 21 with EIA and 25 without EIA, referred to as “asthma controls” hereafter. Initial analysis involved all patients regardless of ICS use. In subsequent analysis, it was determined whether ICS use might modify the effect of EIA status on MF values. The rationale was that ICS use suppresses airway inflammation and could therefore suppress breath compounds related to EIA. It is reasonable to expect this as exhaled nitric oxide suppression by steroids is a well established effect in asthma.

Consequently, the relationship between MFs and EIA status in the ICS negative patients might be expected to be stronger. A two-way ANOVA was used to test for ICS effect modification. Two MFs were identified in both the lower and upper breath samples (see table 2). Subsequent analysis showed that these MFs are dodecane and octanal.

TABLE 2
ICS effect modification

| Compound | Breath fraction | ICS effect modification p-value |
|---|---|---|
| dodecane | Lower | 0.02 |
| octanal | Lower | 0.02 |
| dodecane | Upper | 0.004 |
| octanal | Upper | 0.02 |

Prior to running the analysis, a threshold p value of p<2 was established as a cut-off for what would be considered as evidence of interaction

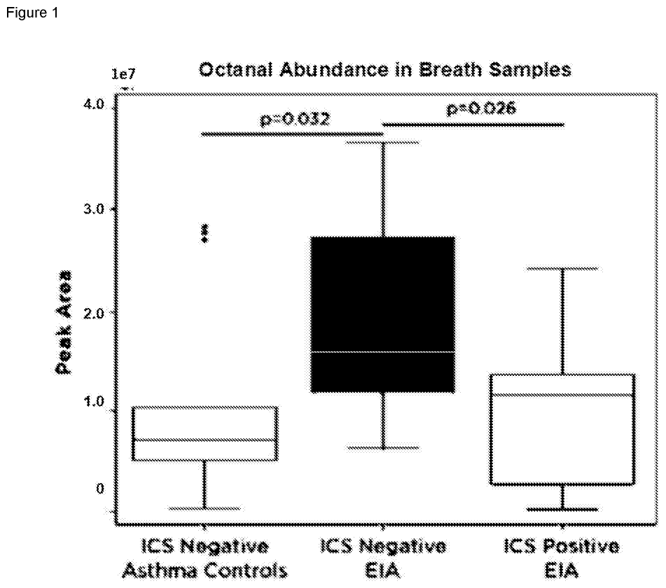

The abundance of dodecane and octanal in breath samples and the effect of ICS modification is shown in FIG. 1 and table 2. FIG. 1 relates to octanal. A similar result was obtained with dodecane. The p value for ICS in EIA group (right box value as in FIG. 1) for dodecane is 0.1697; for the left box it is 0.01.

Importantly, these data indicate a biomarker elevated in untreated individuals with asthma whilst the same biomarker is absent in those without asthma and clearly suppressed to normal levels in individuals treated with the eosinophilic inflammation suppressing drug ICS.

### CONCLUSIONS

This study analysed breath from EIA patients and asthma patients and the effect of ICS treatment breath compounds.

This study identified biomarkers which reflected the presence of untreated EIA. It was shown that ICS treatment suppressed the change in the VOCs dodecane and octanal associated with EIA returning them to normal levels indicating its association with airway inflammation (see FIG. 1 and table 2).

1. An in vitro method of predicting the response of a subject suffering from a respiratory disorder to a therapy comprising a Th2 pathway modulator comprising the steps of
determining the amount of dodecane and/or octanal in a sample of exhaled breath of said subject;
comparing the amount of dodecane and/or octanal to a reference value and
predicting that the patient will respond to the therapy when the amount measured in the sample is different compared to the reference level.

2. An in vitro method of monitoring the efficacy of a treatment of a subject suffering from asthma or a respiratory disorder with a therapy comprising a Th2 pathway modulator comprising the steps of
determining the amount of dodecane and/or octanal in a sample of exhaled breath of said subject;
comparing the amount of dodecane and/or octanal to a reference value and
predicting that the patient will respond to the therapy when the amount measured in the sample is different compared to the reference level.

3. The method of claim 1 or 2 wherein the reference value is from the same subject and the method predicts exacerbation risk in patients undergoing treatment, for example with ICS.

4. The method of any of claims 1 to 3 wherein the respiratory disorder is asthma.

5. The method of any one of the preceding claims wherein the respiratory disorder is eosinophilic airway inflammation.

6. The method of any one of the preceding claims wherein the Th2 pathway modulator is an inhibitor.

7. The method of any one of the preceding claims wherein the Th2 pathway inhibitor is a steroid.

8. The method of any one of the preceding claims wherein the reference level is from a subject suffering from a respiratory disorder that has not been treated with the Th2 pathway modulator comprising predicting that the patient will respond to the therapy when the amount measured in the sample is elevated compared to the reference level.

9. The method of any one of the preceding claims wherein the reference level is the median level of the respective marker in a reference population.

10. The method of any one of the preceding claims wherein the sample is a lower and/or upper breath sample.

11. The method of any one of claims 1, 2 or 4 to 10 wherein the subject is not undergoing therapy with inhaled corticosteroids.

12. The method of any one of the preceding claims further comprising establishing a reference value in a reference subject.

13. The method of any one of the preceding claims further comprising selecting a treatment or treatment dosage.

14. An in vitro method of diagnosing, prognosing and/or monitoring a respiratory disorder in a subject comprising the steps of:
determining the amount of dodecane and/or octanal in a sample of exhaled breath of said subject;
comparing the amount of dodecane and/or octanal to a reference value and
predicting that the patient is likely to suffer from a respiratory disorder when the amount measured in the sample is different compared to the reference level.

15. The method of claim 14 wherein the respiratory disorder is asthma.

16. The method of claim 14 wherein the respiratory disorder is eosinophilic airway inflammation.

17. The method of claims 14 to 16 wherein the reference level is from a subject suffering from a respiratory disorder that has not been treated with the Th2 pathway modulator comprising predicting that the patient will respond to the therapy when the amount measured in the sample is elevated compared to the reference level.

18. The method of any one of claims 14 to 17 wherein the reference level is the median level of the respective marker in a reference population.

19. The method of any one of claims 14 to 18 wherein the sample is a lower and/or upper breath sample.

20. The method of any one of claims 14 to 19 wherein the subject is not undergoing therapy with inhaled corticosteroids.

21. The method of any one of claims 14 to 20 further comprising establishing a reference value in a reference subject.

22. An in vitro method of distinguishing eosinophilic airway inflammation in a subject from other types of airway inflammation comprising the steps of:
determining the amount of dodecane and/or octanal in a sample of exhaled breath of said subject;
comparing the amount of dodecane and/or octanal to a reference value and
predicting that the patient is likely to suffer from eosinophilic airway inflammation when the amount measured in the sample is elevated compared to the reference level.

23. The method of claim 22 wherein the reference level is from a subject suffering from eosinophilic airway inflammation that has not been treated with the Th2 pathway modulator comprising predicting that the patient will respond to the therapy when the amount measured in the sample is elevated compared to the reference level.

24. The method of claims 22 to 23 wherein the reference level is the median level of the respective marker in a reference population.

25. The method of any one of claims 22 to 24 wherein the sample is a lower and/or upper breath sample.

26. The method of any one of claims 22 to 25 wherein the subject is not undergoing therapy with inhaled corticosteroids.

27. The method of any one of claims 22 to 26 further comprising establishing a reference value in a reference subject.

28. The method of any one of the preceding claims further comprising selecting a treatment for said subject.

29. A method of treating asthma or a respiratory disorder in a patient, comprising administering to the patient a therapeutically effective amount of a Th2 pathway inhibitor, wherein an exhaled breath sample obtained from the patient has been determined to have elevated levels of dodecane and/or octanal, compared to reference levels of dodecane and/or octanal.

30. A device for use in a method of any of claims 1 to 29.

31. A VOC selected from dodecane and/or octanal for use in diagnosing, prognosing and/or monitoring a respiratory disorder.

32. A VOC according to claim 31 wherein the respiratory disorder is asthma.

33. A VOC according to claim 32 wherein the respiratory disorder is eosinophilic airway inflammation.
